(19) United States
(12) Patent Application Publication
(10) Pub. No.: US 2021/0003528 A1
(43) Pub. Date: Jan. 7, 2021

(54) HANDHELD SENSOR FOR RAPID, SENSITIVE DETECTION AND QUANTIFICATION OF SARS-CoV-2 FROM SALIVA

(71) Applicant: University of Florida Research Foundation, Inc., Gainesville, FL (US)

(72) Inventors: Josephine F. Esquivel-Upshaw, Gainesville, FL (US); Fan Ren, Gainesville, FL (US); Stephen J. Pearton, Gainesville, FL (US); Steven Craig Ghivizzani, Gainesville, FL (US); Samira Afonso Camargo, Gainesville, FL (US); Chaker Fares, Gainesville, FL (US); Minghan Xian, Gainesville, FL (US); Patrick H. Carey, Gainesville, FL (US); Jenshan Lin, Gainesville, FL (US); Siang-Sin Shan, Hsinchu City (TW); Yu-Te Liao, Hsinchu City (TW); Shao-Yung Lu, Hsinchu City (TW)

(21) Appl. No.: 17/025,664

(22) Filed: Sep. 18, 2020

Related U.S. Application Data
(63) Continuation-in-part of application No. 16/851,859, filed on Apr. 17, 2020.
(60) Provisional application No. 62/835,962, filed on Apr. 18, 2019.

(51) Int. Cl. G01N 27/414; B01L 3/00; C12Q 1/6825

(52) U.S. Cl. CPC G01N 27/4145; B01L 3/50857; C12Q 1/6825; G01N 27/4146; B01L 2300/16; B01L 2300/0663; B01L 2300/0819; B01L 2300/0893; B01L 2300/0636

(57) ABSTRACT

Various examples are provided for disposable medical sensors that can be used for detection of SARS-CoV-2 antigen, cardiac troponin I, or other biosensing applications. In one example, a medical sensing system includes single-use disposable test strip comprising a functionalized sensing area configured to detect SARS-CoV-2 antigen and a portable sensing and readout device including pulse generation circuitry that can generate synchronized gate and drain pulses for detection and quantification of SARS-CoV-2 antigen in biological samples. In another example, a method includes providing a saliva sample to a functionalized sensing area configured to detect SARS-CoV-2 antigen, generating synchronized gate and drain pulses for a transistor, the gate pulse provided via electrodes of the functionalized sensing area, and sensing an output of the transistor that is a function of a concentration of SARS-CoV-2 antigen in the sample.

### CROSS REFERENCE TO RELATED APPLICATIONS

This application is a continuation-in-part of co-pending U.S. non-provisional application entitled “Modularized Inexpensive Detection of Cerebral Spinal Fluid for Medical Applications” having Ser. No. 16/851,859, filed Apr. 17, 2020, which claims priority to, and the benefit of, U.S. provisional application having Ser. No. 62/835,962, filed Apr. 18, 2019, both of which are hereby incorporated by reference in their entireties.

### BACKGROUND

The phase of easing restrictions and the “re-opening” in various states and countries will require frequent testing and re-testing of as many individuals as possible (health care workers in particular) for the foreseeable future to sustain viral containment. Addressing this persistent problem will use new technologies beyond those that are currently available. The new technologies should be inexpensive and readily accessible. Most importantly, the systems should provide rapid and dependable readouts to prompt immediate identification and isolation of those carrying Sudden Acute Respiratory Syndrome Coronavirus 2 (SARS-CoV-2). Thus, there is a need for development of an easy-to-use non-laboratory handheld test device capable of testing samples in real time without involving trained technician.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 illustrates an example of the variation in Debye length, λ_{D}, in solutions with different ion concentrations, in accordance with various embodiments of the present disclosure.

FIG. 2 illustrates an example of schematic diagrams illustrating single-pulse and double-pulse sensing methods, in accordance with various embodiments of the present disclosure.

FIG. 3 illustrates an example of a double-layer structure utilizing charge induction to reduce the screening effect, in accordance with various embodiments of the present disclosure.

FIG. 4 is a block diagram illustrating an example of a device for double-pulse sensing, in accordance with various embodiments of the present disclosure.

FIG. 5 is a schematic diagram illustrating an example of circuitry for an integrated sensing and readout device, in accordance with various embodiments of the present disclosure.

FIGS. 6-9 are schematic diagrams illustrating examples of circuitry of the integrated sensing and readout device of FIG. 5, in accordance with various embodiments of the present disclosure.

FIGS. 10A and 10B are a schematic diagram and image of an example of an integrated sensing and readout device, in accordance with various embodiments of the present disclosure.

FIG. 11 includes images of a prototype of a handheld sensing and readout device, in accordance with various embodiments of the present disclosure.

FIG. 12A and 12B are a block diagram illustrating an example of a sensing device configured for the simultaneous or sequential testing of multiple sensors and is an image of an 8 channel testing system used for simultaneous multiple-testing with the UBS connectors to avoid the contamination of the test unit, in accordance with various embodiments of the present disclosure.

FIG. 13 includes images of an example of a testing strip and the tip area, in accordance with various embodiments of the present disclosure.

FIG. 14 illustrates an example of SARS-CoV-2 functionalization, in accordance with various embodiments of the present disclosure.

FIG. 15 illustrates an example of a test strip including reference and active sensing areas, in accordance with various embodiments of the present disclosure.

FIG. 16 is a schematic illustrating an example of a test strip comprising microfluidic channels configured with a plurality of sensing electrodes, in accordance with various embodiments of the present disclosure.

FIGS. 17A and 17B illustrate examples of measurement results for SARS-CoV-2 from saliva, in accordance with various embodiments of the present disclosure.

FIGS. 18A-18D illustrate examples of measurement results for troponin I, in accordance with various embodiments of the present disclosure.

### DETAILED DESCRIPTION

Disclosed herein are various embodiments related to handheld sensors for rapid and sensitive detection and quantification of conditions from saliva. Currently, the most commonly used method for detecting potential diseases is to test body fluids, such as serum urine, and blood, using biochemical analysis. However, when the biomarker is in a high-concentration ionic solution, the measurement results may be severely affected by shielding effects, or so-called screening effects. Reference will now be made in detail to the description of the embodiments as illustrated in the drawings, wherein like reference numbers indicate like parts throughout the several views.

Biosensor technologies have progressed to achieve high sensitivity and extended dynamic range over a wide concentration range of biomarkers. Semiconductor-based sensors, such as the ion-sensitive field-effect transistor (ISFET), biologically functionalized field-effect transistor (BioFET), and DNA field-effect transistor (DNAFET), can provide reliable and fast measurements for composition analysis by functionalizing the transistor's gate electrode with antibodies to detect antigens in a solution. However, in a highly ionic concentration solution such as saliva, the Debye length is shortened.

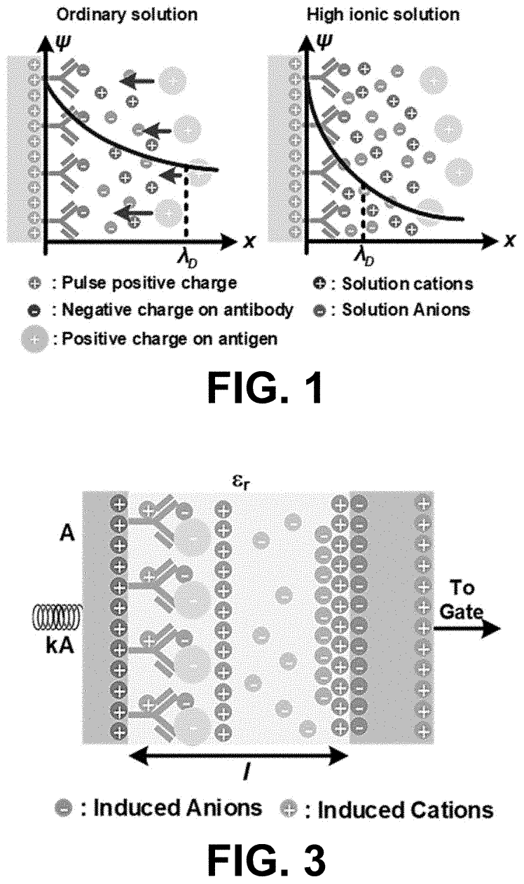

FIG. 1 illustrates an example of the variation in Debye length, λ_{D}, in solutions with different ion concentrations. Debye length is defined as the physical distance that an electrical field generated by a charge can act on neighboring charged particles/molecules. As shown in FIG. 1, the Debye length becomes shorter as the ionic strength increases. Therefore, in a high ionic concentration solution, the Debye length is short, and thus, fewer particles can be attracted or repulsed, making it difficult for the detection antibodies and target antigens to interact and generate a stable potential. In addition, an unstable potential is induced by many free antigens at the gate of the FET under high-concentration environments, which leads to a lower sensitivity and unbalanced output current fluctuations. However, functioning in highly ionic solutions is important for real-time non-laboratory testing, at home or in the field, without the need to reduce ion concentrations through sample dilution.

To overcome the above challenges, two techniques have been developed. First, a double-layer structure utilizes charge induction to reduce the screening effect. The double-layer sensor architecture features a gate electrode separated from the solution to give a stable voltage to the gate of the transistor, performing voltage to current conversion and utilizing charge induction to reduce the screening effect. It also allows the sensing area to be physically separated from a handheld device housing the sensing and readout circuits. An electrical double layer (EDL) gated FET biosensor that is capable of direct detection of target analytes in solutions has been developed. The disposable sensor chip can include a substrate, sensing lines, passivation layer and cap layer. The electrode can be functionalized for sensing, and a window or opening can allow visualization of a sample applied to the functionalized electrode. U.S. non-provisional application entitled “Modularized Inexpensive Detection of Cerebral Spinal Fluid for Medical Applications” having serial no. 16/851,859, filed Apr. 17, 2020, which is hereby incorporated by reference in its entirety, provides additional information about the biosensor.

The second technique, called a synchronized double-pulsed method, ensures that the sensor transistor and circuits are reset before each measurement so that accurate test results can be obtained with high sensitivity in each test. In a conventional test method with a constant bias voltage on the transistor's drain, the charges will accumulate on the drain and affect sensor accuracy. Instead of applying a constant bias voltage, synchronized double short pulses are applied to both drain and gate electrodes of sensor transistor to ensure that the sensor electrode is reset before each measurement without accumulating charges from previous measurements.

### Double-Pulsed Sensing Mechanism

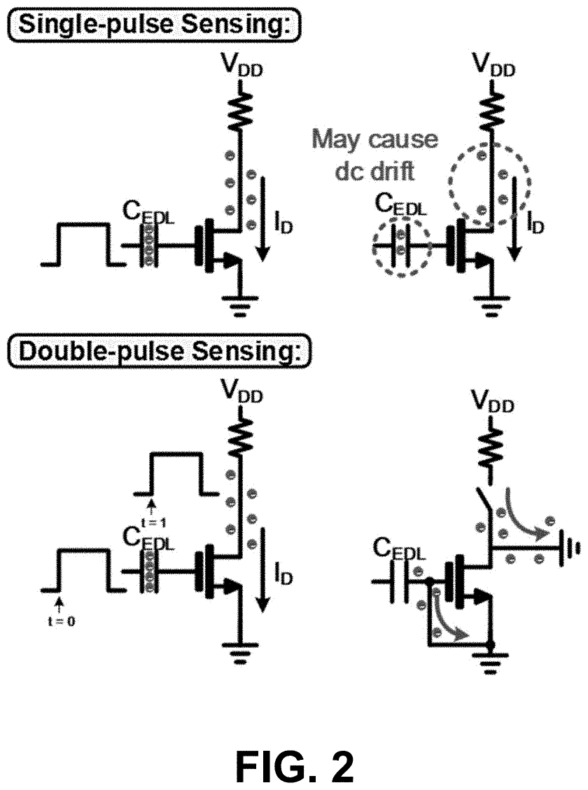

In a single-pulse readout circuit, charges may accumulate on the gate and drain after each measurement because of the floating-gate effect. FIG. 2 graphically illustrates the charge accumulation using single-pulse and double-pulse sensing methods. Without resetting the gate in single-pulse sensing, the accumulated charge shifts the gate/drain voltage in each measurement and results in error. By utilizing a double-pulse technique, the drain and gate paths are sequentially activated to mitigate the charge accumulation at the gate and drain of the sensing transistor.

In electrolytes, Debye length describes the distance at which a charge can act on another charge effectively. Assuming the electrolyte is monovalent and symmetrical, the Debye length can be derived as

$${λ}_{D} = \sqrt{\frac{{ɛ}_{r} {ɛ}_{0} R T}{2 \times 1 {0}^{3} {F}^{2} {C}_{0}}} , \qquad ( 1 )$$

where λ_{D} is Debye length, ϵᵣ is the dielectric constant, ϵ₀ is vacuum permittivity, R is the gas constant, F is the Faraday constant, T is absolute temperature, and C₀ is electrolyte concentration in molarity (M). The Debye length can be also calculated from conductivity, σ

$${λ}_{D} = \sqrt{\frac{ɛ D}{σ}} , \qquad ( 2 )$$

where ϵ is the dielectric permittivity and D is the diffusion coefficient. When the pulse is applied to the electrode, the double-layer forms at the solution-electrode interface and creates a double-layer capacitance, which is correlated with the electrolyte concentration. In an isolated system where the net plate charge is fixed, the lateral redistribution of the surface charge density coupled to the transverse reorganization of the ionic charge distribution in the electrolyte can cause the EDL capacitance to vary.

A relaxing gap capacitor can be used to model the effects of the varying capacitance. FIG. 3 shows an example of a double-layer capacitor model. The EDL capacitance varies with charge diffusion in the solution until charge equilibrium is reached. This phenomenon can be further explained by Hooke's Law. Therefore, on a microscopic scale, the electrostatic energy of the capacitor for a fixed surface charge density, d, can be derived as:

$$E ( d , l ) = \frac{{Ad}^{2} l}{2 {ɛ}_{r} {ɛ}_{0}} + \frac{k A}{2} {( l - {l}_{0} )}^{2} , \qquad ( 3 )$$

where A is the plate area, l₀ is the initial distance between two plates, and k is the spring constant. By Hooke's Law, spring length is determined by:

$$l = - \frac{\vec{F}}{k} , \qquad ( 4 )$$

Assuming that the whole system is in the damped simple harmonic motion, lcan be derived as:

$$l ( t ) = {l}_{0} + {A}_{0} {e}^{\frac{- bt}{2 m}} cos ( {ω}^{′} t + ϕ ) , \qquad ( 5 )$$

where ω is the angular frequency, and φ is the phase.

According to the above equations, in a fixed analyte concentration, the damping time to steady-state corresponds to the capacitance and conductivity of the solution. The relative dielectric constant (ϵᵣ) of the solution decreases as the ionic concentration increases. Besides, the Debye length is inversely correlated with the ionic concentration, resulting in an increase in the EDL capacitance as the conductivity increases. Therefore, the capacitance can be denoted as:

$${C}_{E D L} = \frac{2 E ( d , l )}{{V}_{pulse}^{2}} , \qquad ( 6 )$$

where Vₚᵤₗₛₑ² is the amplitude of the gate pulse pattern sent to the test strip.

On a macroscopic scale, when the electrostatic energy becomes stable in the capacitor, the final DC value of the gate voltage can be determined by the ratio of the EDL capacitance and parasitic capacitance, C_{GS} and C_{GD}. Therefore, for different concentrations of analytes, the gate voltage varies because of the capacitive voltage divider. Theoretically, the analytes can be assessed by measuring the voltage waveform pattern at the gate terminal; however, to avoid other charge leakages affecting measuring results, the drain current, which is amplified by the current mirror gain, is used to assess the analytes in the proposed system.

As observed from the waveform of the sensing electrodes, the analyte information can be in the form of the settling response and the stable voltage of the gate terminal. Measuring the voltage in a stable state can give precise results, and the data recorded from the settling response can provide substantial gain and kinetic dynamics for the charge reaction between the solution and electrode.

The disclosed technology may be used to provide non-laboratory testing with a degree of precision that can avoid complications resulting from current tests being performed only at limited sites due to cost and expertise. The sensing electrode can be fabricated on a disposable strip and can be utilized with a handheld device for data processing and storage. This non-laboratory device can detect a wide range of biomarkers by inserting the specific disposable testing strip into the associated handheld device or an extension cable connecting to the handheld device. An optional detachable connection such as a cable (e.g., USB cable) can be used between the disposable test strip and the portable (or handheld) sensing and readout device; serving as an extension to further separate the test strip and the sensing and readout device, for better sanitization process or other purposes. The ability to function in highly ionic solutions is important if whole blood, urine, nasal secretions, saliva, etc. are to be analyzed at the non-laboratory testing location. U.S. non-provisional application entitled “Low Cost Disposable Medical Sensor Fabricated on Glass, Paper or Plastics” having Ser. No. 16/206,493, filed Nov. 30, 2018, demonstrates the use of this concept to measure the low volume (10 to 50 μl) of Zika virus and U.S. non-provisional application entitled “Modularized Inexpensive Detection of Cerebral Spinal Fluid for Medical Applications” having Ser. No. 16/851,859, filed Apr. 17, 2020, demonstrates its use for the detection of cerebral spinal fluid (CSF), both of which are hereby incorporated by reference in their entireties, but the sensors and methods can be extended to include detection and quantification of SARS-CoV-2, troponin I protein (heart attack detection), or other biomarkers such as, e.g., MRSA or HIV in high ionic solutions.

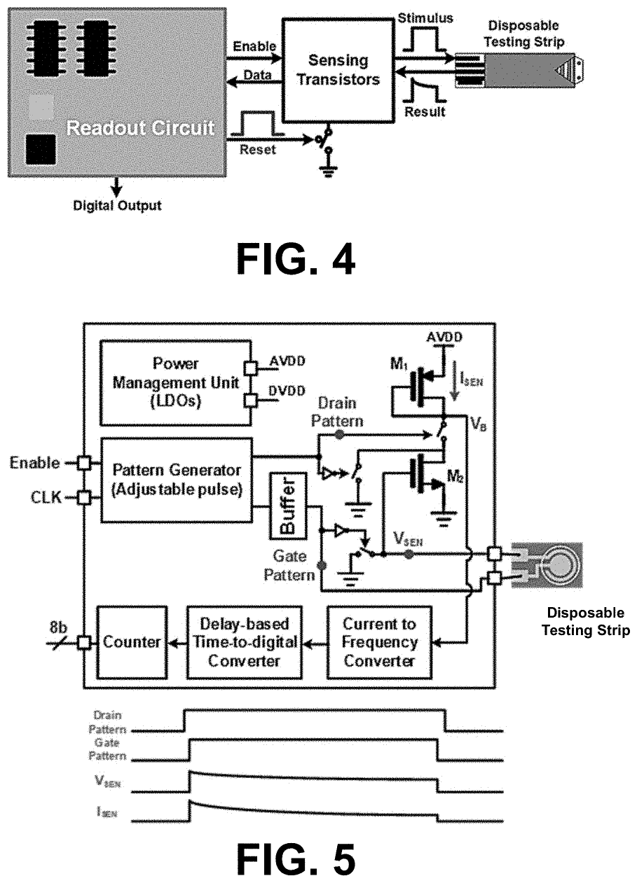

This synchronized double-pulsed method in combination with the double-layer structure allows the test strip sensing area to be externalized from the sensor electronics in the handheld device, and an optional extension cable similar to USB cable can be used to further separate test strip from the handheld device (test strip inserted into one end of extension cable whereas the other end of extension cable is inserted into the handheld device). FIG. 4 is a block diagram illustrating the synchronized double-pulsed method being used with a disposable testing strip. This strip-based platform sensor, similar to glucose detection, can use a functionalized disposable strip with, e.g., antibody protein to detect the SARS-CoV-2 spike protein or target antibodies for troponin I measurement, among others. After testing, a new unused low-cost testing strip can be inserted into (or coupled to) the device after the removal and disposal of the used testing strip. The test strip can be functionalized with the antibody for a targeted virus on, e.g., a glass strip or plastic strip, that can be electrically connected to the gate of the FET. It can also reduce the detection time to less than 3 sec. The sensing and readout circuitry used for the measurement will now be presented.

Target detection is based upon the selective recognition and interaction of two protein molecules (akin to a receptor protein binding to its cognate ligand), such that either the receptor or ligand is immobilized on the test strip electrode and serves as the “detection” protein, while the second protein, suspended in a biological fluid (e.g. blood, urine, saliva etc.), serves as the “target.” When a test solution containing the target is applied to the test strip, the selective recognition and binding (complex formation) between the target and immobilized detection protein, changes the charge distribution on the test electrode in response to an electrical pulse, which is then amplified to the output of the transistor.

This principle was demonstrated with the use of a receptor (anti-SARS-CoV-2 spike antibody) as the “detection” protein and its ligand (SARS-CoV-2 spike protein) as the “target.” In this configuration (#1) the test system is used to detect the presence of SARS-CoV-2 spike antigen (and by extension the SARS-CoV-2 virus) in the saliva of test subjects. The converse configuration (#2) is also possible, whereby the ligand of the pair (e.g. the SARS-CoV-2 spike protein) is immobilized on the electrode and is used to “detect” the presence of a receptor protein (e.g. anti-SARS-CoV-2 spike antibody) in a biological fluid. In configuration #2, the system is used to detect the presence of anti-SARS-CoV-2 antibody in the blood, or serum, of test subjects, indicative of a humoral immune response from prior SARS-CoV-2 infection.

### Sensor and Readout Architecture

FIG. 5 shows a block diagram and the output pulse patterns of the sensing and readout circuits including, e.g., an adjustable pulse pattern generator, a current-to-frequency (I-to-F) converter, and a delay-line-based time-to-digital converter (TDC). The pattern generator can generate two adjustable pulses for the drain and gate of the transistor, which operates as a transconductance device for voltage to current. The pulse width (duration) and voltage amplitude can be adjusted to optimize the detection sensitivity.

As the gate pulse passes through the test strip sensing area and analyte, Vₛₑₙ varies according to the double-layer capacitance on the electrode-solution interface until the energy stored in the double-layer capacitor remains stable. The corresponding voltage at the gate terminal of the NMOS transistor creates the drain current, Iₛₑₙ, which is detected by the following I-to-F converter. The change in Iₛₑₙ results in a change in the output frequency of the oscillator. Then, a delay-line based TDC converts the I-to-F outputs into digital codes that represent the analyte concentration.

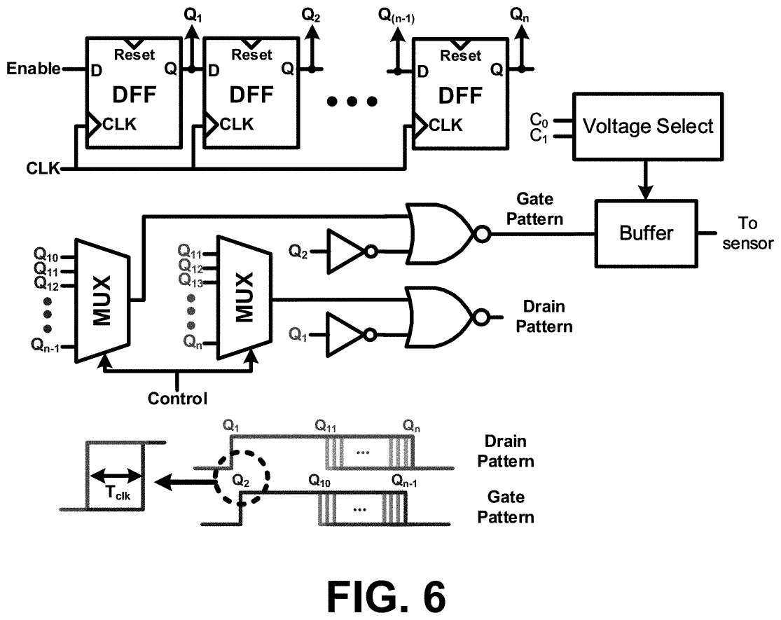

Pulse Pattern Generator. FIG. 6 shows a schematic of an example of the pulse pattern generator. A programmable shift register chain can control the delay between the drain and gate pulse patterns. In the generator, one input of each NAND gate can be connected to Q₁ and Q2 of the D-flip-flops, respectively; therefore, a time delay with T_{clk} is generated between the rising edge of two clock signals. For falling edges, two 16-to-1 multiplexers (MUXs) with the same control signal can be used. Two MUX inputs can be shifted for choosing T_{clk}. For example, when the control bit is 0000, Qio and Qii will be sent to the output; when the control bit is 1111, Qₙ₋₁ and Qₙ will be sent to the output. By this method, a time delay for T_{clk} can be created between two falling edges of the signals.

The gate and drain pulses and the pulse delay between them can be reconfigured for different sensors or different concentrations. For example, when measuring low-concentration troponin I, the response is often long, so an extended pulse signal can be used to provide sufficient time to acquire steady state information. For measuring other analytes with a faster response, the pulse pattern generator can create short pulses to increase the sampling rate. In the design, the drain pulse pattern can be adjusted from 10T_{clk} to (n−1)T_{clk}, and the gate pulse pattern can be adjusted from 8T_{clk} to (n−3)T_{clk}, where n is the number of delay cells.

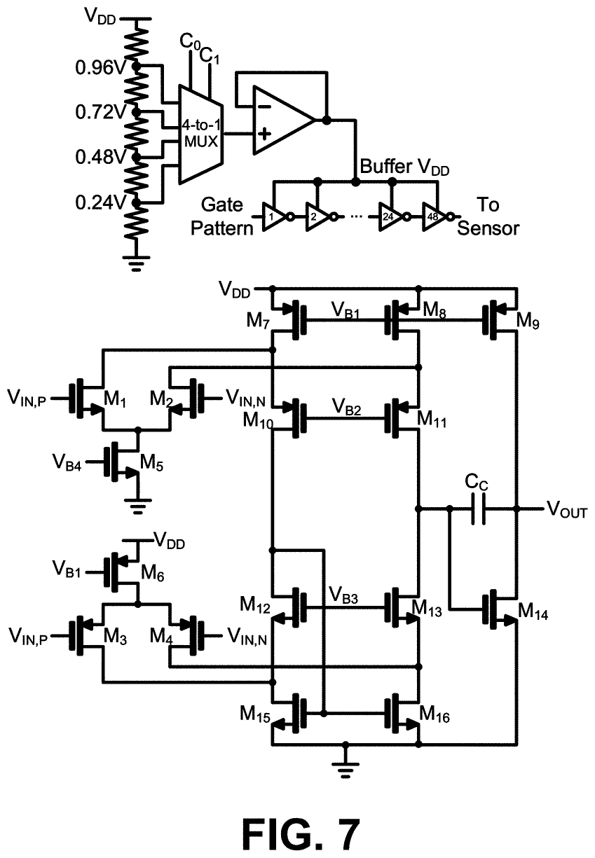

In order to detect various substances, which may have different reaction potentials, the sensing and readout circuits can be configured to generate an accurate voltage to stimulate the chemical reaction. In the pulse pattern generator, a V_{DD}-adjustable buffer can be implemented to adjust the amplitude of the gate pulse pattern. A schematic diagram of the V_{DD} adjustment circuit (generator and buffer) is shown in FIG. 7. It mainly includes a resistor chain, 4-to-1 MUX, and folded-cascoded differential-to-single-ended amplifier. The resistor chain generates four different voltages, and the output voltage can be controlled by two external signals, C₀ and C₁. To ensure that the amplifier can work over a wide range in voltage, a complementary input stage is employed. The second stage of the amplifier provides extra amplification and voltage swing. To supply enough power for the buffer, the W/L ratio of two transistors (M₉ and M₁₄) in the second stage is about 40× larger than that in the first stage. The output of the unity-gain amplifier is applied to the inverter buffer stages to adjust the pulse amplitude. Wth the adjustable pulse amplitude and interval, the design is suitable for a wide range of sensing applications.

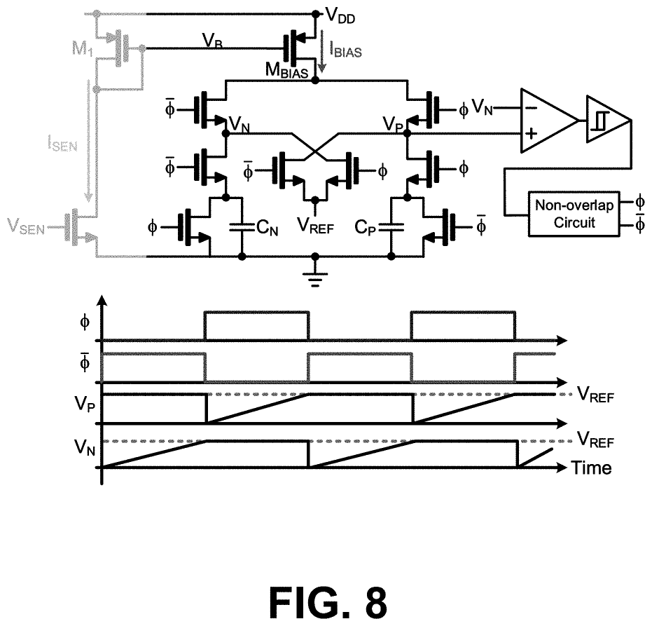

Current to Frequency Converter (I-to-F). FIG. 8 shows a schematic and timing diagram of an example of the I-to-F circuit. The I-to-F circuit can comprise two charging/discharging paths, an analog comparator, and a Schmitt-trigger. When ϕ is high, I_{BIAS} starts to charge C_{P} while C_{N} starts to discharge, and V_{N} connects to V_{REF}. As the voltage, V_{P}, is charged and becomes higher than V_{REF}, the comparator changes the state, making ϕ change from high to low and swapping the inputs of the comparator. Generally, the comparator offset affects the accuracy of the I-to-F converter. In this structure, the comparator offsets can be compensated by swapping the comparator's inputs in the charging and discharging states, resulting in the overall period remaining unchanged.

To sense the drain current generated by Vₛₑₙ, a current mirror is implemented. Therefore, I_{BIAS} can be written as:

I_{BIAS}=αl_{SEN}=αβ(V_{SEN}−V_{th,n})² (7)

where α is the size ratio between M₁ and M_{BIAS} in FIG. 8, pis the transconductance coefficient, and V_{th,n} is the threshold voltage of the sensing transistor (M₂) in FIG. 4. The duration of the output square wave (Tₒᵤₜₚᵤₜ) can be derived as follows:

$${T}_{output} = \frac{( {C}_{N} + {C}_{P} ) {V}_{R E F}}{{I}_{BIAS}} = \frac{( {C}_{N} + {C}_{P} ) {V}_{R E F}}{α {β ( {V}_{S E N} - {V}_{th , n} )}^{2}} , \qquad ( 8 )$$

with the relation between I_{SEN} and Iₒᵤₜₚᵤₜ, the concentration of the analyte can be obtained.

Delay-line-based Time-to-digital Converter (TDC). In the TDC design, the limit of detection for the counter-based TDC can be determined by the clock speed, leading to tradeoffs among power, area, and resolution. The delay-line-based TDC has the advantage of first-order noise shaping, and the minimum delay time of each block determines the resolution, which benefits from the advanced technology node. Thus, the delay-line TDC can achieve a better resolution, smaller area, and lower power consumption than the counter-based TDC.

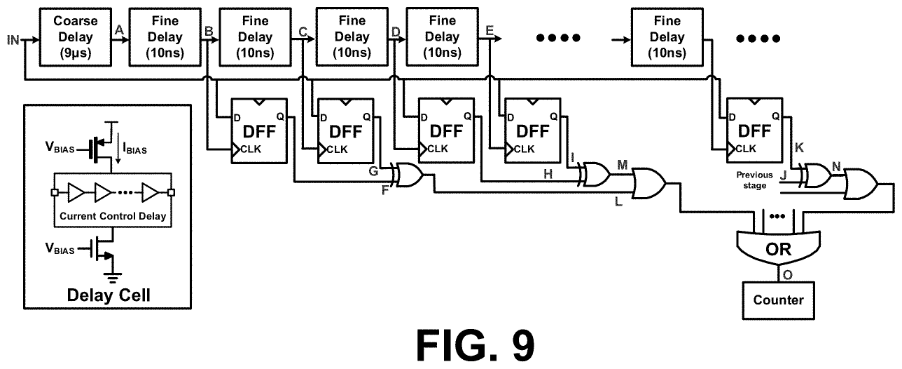

FIG. 9 shows a schematic diagram of an example of the TDC. The delay-line TDC comprises a plurality of current-starved inverters to create the delay steps. A coarse delay block with a 9-μs delay can be employed to reduce the number of bits and silicon area. Afterward, the signal can be digitalized by a fine delay chain with a 10-ns delay in each stage. To detect the falling edge of the input signal, each clock input of the DFF can be connected to the delayed signal, and the sensing signal can be connected to the input of the DFF. When the signal has not been delayed for a positive duration, the output of the DFF is pulled from low to high (F, G, H, I, J) sequentially. Every two DFFs share an XOR gate, and the clock signal for the counter can be generated (L, M) by implementing the XOR gate to the output signals. When the DFF detects the falling edge of the original signal, the output of the DFF remains at a low level and creates the last clock signal for the counter (N). Finally, the duration of each I-to-F output represents the concentration of the analyte. The sampling signals are summed by an 8-bit counter. The counter encodes the duration into a series of digital codes. In addition, the counter acts as a low-pass filter to suppress the high-frequency fluctuations in the measurement.

### Handheld Device and Testing Strip

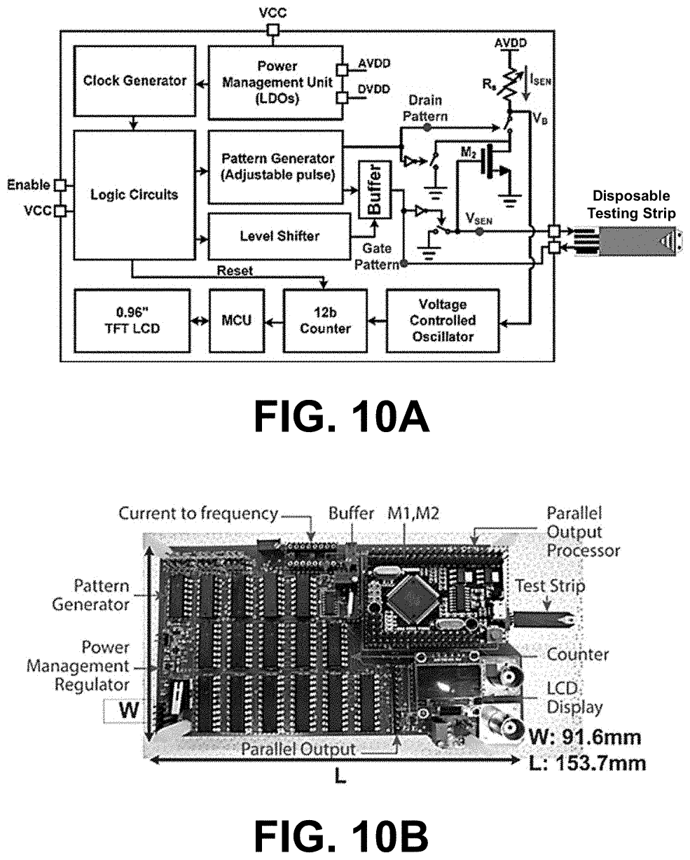

Referring to FIGS. 10A and 10B, shown is a block diagram and an image of a prototype of the sensing and readout circuits, respectively, were designed and used to produce electrical pulses to the disposable testing strip, measuring the responses by the on-board Si-MOSFET, amplifying the signals from the strip, and converting the amplified signal to an analog or digital output. The sensing and readout circuits can be used in a handheld device that can display the results allowing non-laboratory (e.g., point-of-care or over-the-counter) testing.

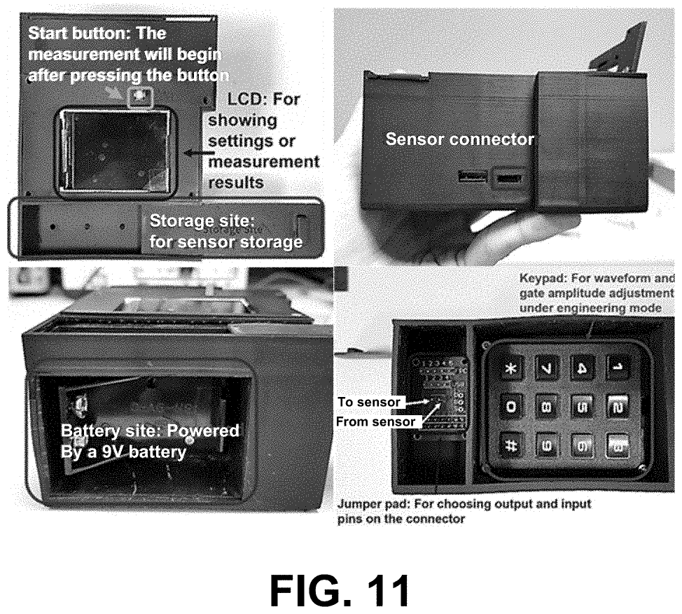

FIG. 11 shows a prototype of the handheld device. The image in the upper left shows the front side of the handheld device comprising an LCD display to show the test result or setting of the device, a storage compartment for spare strips and a button to start the test after inserting the strip into the side of the device. The image in the upper right shows the connector side of the handheld device for inserting the test strip. The connector can be a standard-type USB connector or other appropriate connector. The image in the lower left shows another side of the handheld device with a compartment for a 9 V battery. The bottom of the handheld device is shown in the image on the lower right. A keypad and jump pad can be included for adjusting the waveform and amplitude of the signals but may be removed in some embodiments.

The handheld device includes power management units, a reconfigurable pattern generator, a current-to-frequency converter, and digital signal processing units. One of the electrodes of the sensor strip can be connected on the gate of the FET. The voltage variation from the interaction of a detection antibody and target analyte (e.g. host protein, viral antigen, disease biomarker etc.) on the electrodes of the sensor strip modulates the current at the drain of the FET. This detected signal can then be converted to the frequency of an oscillator, whose oscillation frequency depends only on the sensor current and a fixed resistor value. The design is insensitive to the amplitude noise from the devices and voltage supply and therefore can achieve low electrical noise and excellent stability. The frequency signal can be digitized using a frequency counter (e.g., 12-bit). The frequency counter provides first-order noise shaping intrinsically. The design uses digital architecture, thus providing the advantages of high design flexibility, compact size, and low power consumption. The pattern generator stimulates the biosensor with a reconfigurable pulse duration (100-1200 μs), delay time (5-40 μs) between the gate and drain stimuli, counting duration (10-100 μs), and gate amplitude (1.2-1.52V) to optimize the sensor.

A wireless communication module (e.g., Bluetooth®) can be included in the handheld device, for wireless data transmission to a user device such as, e.g., a smartphone, or a computer. Together with an application executed on the smartphone, computer, or other remote or mobile device, the Bluetooth® wireless link can make the handheld device easy to use and compatible with many existing personal devices and equipment.

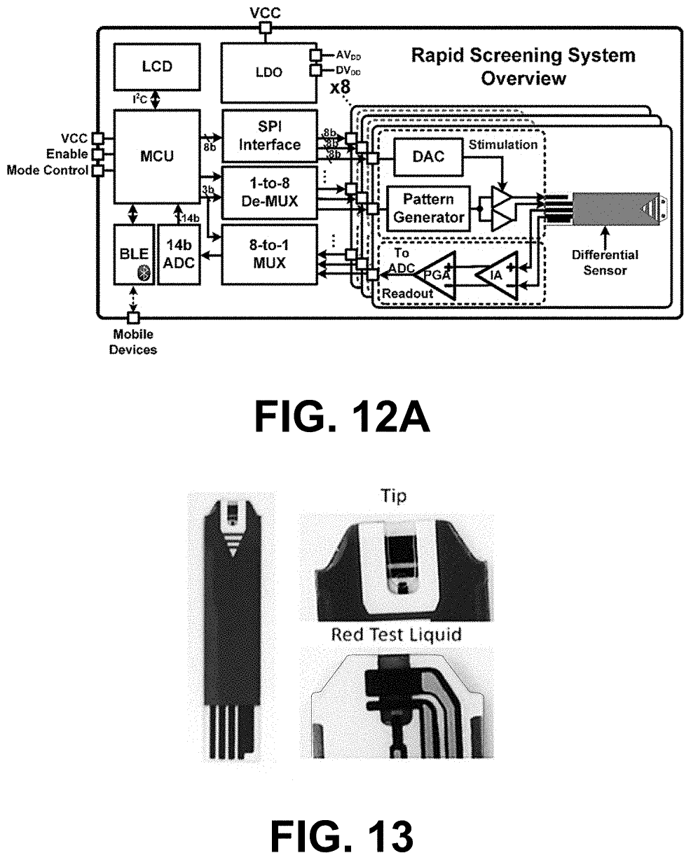

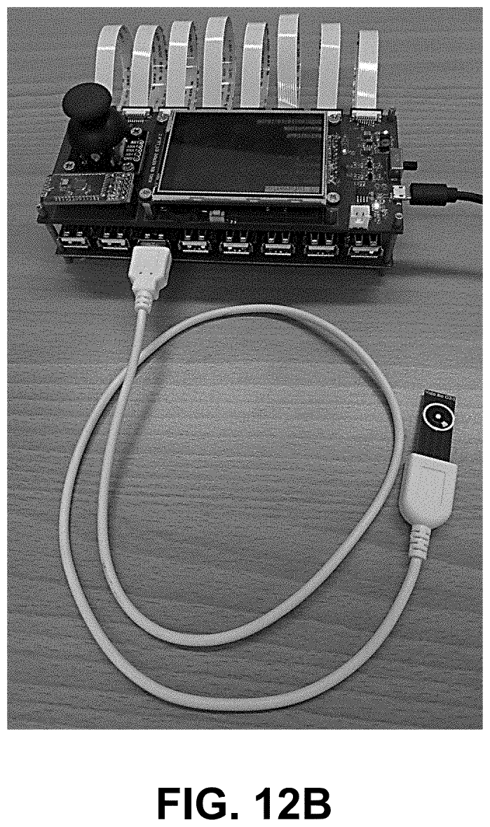

Referring to FIG. 12A, shown is a block diagram illustrating an example of a device configured for the simultaneous or sequential detection analyses of multiple test sensors. Each of multiple differential readout circuits can be connected to sensor electrodes functionalized with detection antibody (e.g., anti-SARS-CoV-2 spike protein antibody, as will be discussed) or reference electrodes for calibration of background noise. The sensor information can be acquired by scanning the readout circuits and recording the data in memory. The microcontroller unit [MCU] can be used to adjust the stimulus pattern and double pulse timing control. Also, the MCU can perform data processing from multiple sensors and communication protocol management. Finally, the data can be transmitted through, e.g., the Bluetooth® wireless link to, e.g., a user device such as a smartphone, computer, or other instrument for data collection and analysis. The design can test multiple sensor strips simultaneously or sequentially to reduce diagnosis or screening time for a large number of individuals.

Wth advanced silicon semiconductor foundry technologies, instead of using off-shelf components on a circuit board, sophisticated biosensor readout circuits can be integrated into a single chip to reduce the size of the handheld devices and significantly improve signal-to-noise ratio by eliminating the wire coupling noise and signal losses. The dimensions of an integrated circuit chip can be much smaller (e.g., 0.66 m×1.37 mm) than the size of a circuit board using off-shelf components. The wire coupling noise and signal loss usually limit the sensitivity of the biosensor readout frontends, especially for very weak bio-signal measurements. Without the additional wiring, the device sensitivity and reliability will also be significantly improved.

In some embodiments, the chip can be fabricated in a 0.18 μm standard CMOS process. To decrease the interference from the environment, the chip can be insulated with epoxy after wire bonding. A chip comprising the pattern generator, I-to-F converter, TDC and 8-bit counter was fabricated in a 0.18 μm standard CMOS process with an area of 0.92 mm². The sensing and readout integrated circuit chip were assembled on a circuit board for functional characterization. The two-electrode sensor can be fabricated using, e.g., an Au surface and modified with antibodies for, e.g., troponin I measurement, SARS-CoV-2 antigen measurement, etc.

The test strips can be made of multiple patterned conductive layers on plastics as the electrodes with either printed silver layer covered with a carbon layer, or gold-based electrodes deposited by, e.g., electric-plating or metal sputter deposition. FIG. 13 includes images of an example of a testing strip and the tip area where the test solution is applied. The tip of the strip has a micro-fluidic channel to draw in the test sample via capillary action, and the quantity of the sample is in the range of 1 to 500 micro-liter, but could be increased for specific test strips and applications.

Although the detection time can be less than 3 sec, the testing can be performed with a delay of, e.g., 30 sec or greater in some applications after applying the sample to the test strip in order to ensure the binding of virus to the antibody. This prototype was designed and demonstrated as an easy-to-use handheld device for individuals at home, health care professionals in their offices and clinics, shops, restaurants, airlines, theaters, sports venues etc. to perform real-time testing and displaying the results. Similar to that shown in FIG. 12A, this platform can be designed in arrays to support multiple strips or a rotative scan system for massive testing of several hundred samples in a batch within minutes for large-scale test facilities handling large quantities of samples. FIG. 12B illustrates an example of an 8-channel testing unit, which can be used to test up to 8 strips simultaneously. The unit also has USC connects to separate test strips from the testing unit to avoid contamination. The unit can be expanded into hundreds or thousands.

### Sensor Functionalization on Testing Strip

The sensor can be functionalized to detect a wide range of targets, depending on conditions. For example, there are antibodies available for targeting the spike, envelope, and nucleocapsid proteins of the SARS-CoV-2 virus. There are antibodies available that target different regions of the spike protein, e.g., one that targets the 17 amino acid sequence near the center of the spike protein, within amino acid 550-600 and another that targets a 20 amino acid sequence near the carboxy terminus, both are available from ProSci Inc.

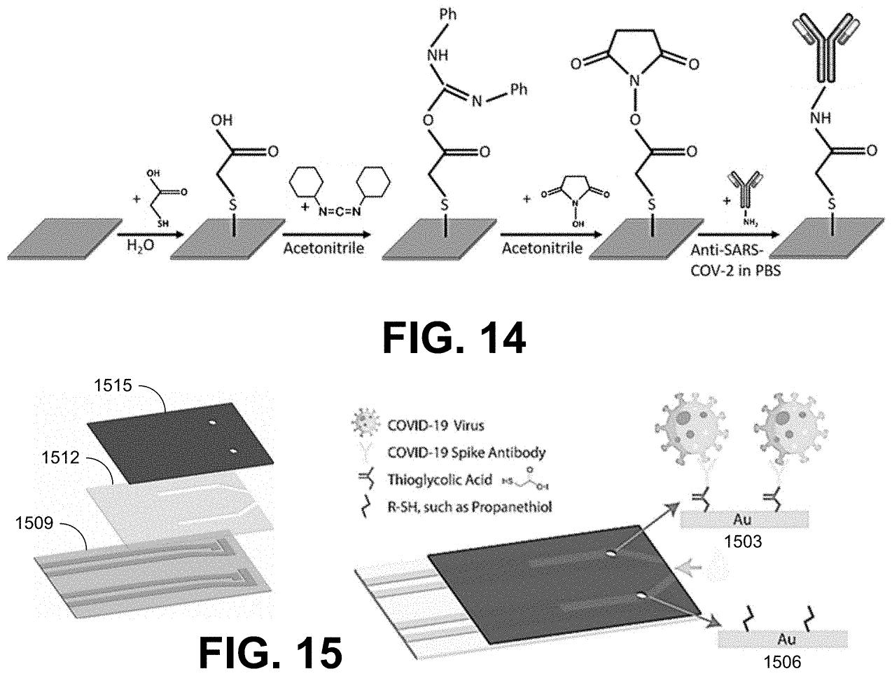

FIG. 14 illustrates an example of test strip electrode functionalization. As shown, an Anti-SARS-CoV-2 antibody can be bound to a gold surface of the electrodes in the sample chamber in the tip of the test strip of the via a chemical reaction that creates a strong chemical bond of thiol to the gold by treating the gold surface with thio-glycolic acid (TGA) for 12 hours at room temperature. Excess TGA can be rinsed off using deionized (DI) water. The Au-S bond will form a strong bond with no additional reagents needed. The tip of the testing strips can then be submerged in a 0.1 mM solution of N,N′-dicyclohexylcarbodiimide in acetonitrile for 30 minutes and then in a 0.1 mM solution of N-hydroxysuccinimide in dry acetonitrile for 1 hour. These functionalization steps result in the formation of succinimidyl ester groups on the gold of the sensing electrodes. The strip with the Anti-SARS-CoV-2 polyclonal antibody (20 μg/mL in phosphate buffer solution (PBS)) can be incubated for 18 hours to affix the antibody on the electrode surface. After these steps, the sensor strip is ready to use. Test-strip functionalization with antibody (or antigen, depending on the application) can be performed by alternate methodologies, e.g. electro-printing, and can be incorporated into test-strip manufacture.

The sensor can also be functionalized for measurement of cardiac troponin I. First, the two electrodes are coated with gold nanoparticles (Vida-Bio, GN3) before post-modification. For surface modification, gold electrodes can be treated with ethanol to remove organic contaminants. The self-assembled monolayers can be formed by immersing the gold electrodes in an ethanolic 10 mM solution of 12-mercaptododecanoic acid (MDA, C₁₂H₂₄O₂S, Sigma-Aldrich) in a sealable container for 18 hours. After that, the MDA-modified gold electrodes can be carefully rinsed and cleaned with ethanol and blown dry with a stream of nitrogen gas. The MDA-modified gold electrodes can then be immersed in an aqueous solution of 10 mg/ml 1-ethyl-3-(3-dimethylaminopropyl) carbodiimide (EDC, Sigma) and 2.5 mg/ml N-hydroxysuccinimide (NHS, Fluka) in citrate buffer solution (pH 5.0) for 1 hour [19]. Then, the MDA-modified gold electrodes can be coated with 100 μg/ml cardiac troponin I antibody (TnI, GeneTex) for 1 hour to immobilize the TnI on the activated MDA-SAM gold electrodes with chemical bonding.

Due to different test subjects or body conditions of the same test subject at different time points, the background sensor output signal may be different. To resolve this issue, a differential sensor can be used which comprises two sets of electrodes and micro-fluidic channels fabricated on the same strip. FIG. 15 illustrates an example of the test strip with a set of reference sensor electrodes 1506 and another set of active sensor electrodes 1503. One set of the electrodes 1506 can be functionalized with propanethiol or other compound with thiol functional groups and without —COOH, —OH or —NH₂ to terminate the electrodes and serve as the reference sensor. The other set of electrodes 1503 can be functionalized with, e.g., anti-SARS-CoV-2 spike protein antibody. The voltage difference between the reference electrodes 1506 and active electrodes 1503 can be amplified via a differential amplifier, and the signal will be converted into digital output using, e.g., an analog-to-digital converter (ADC) or a current to frequency converter followed by a time-to-digital converter (TDC) to convert to digital data for processing. In one configuration, the differential sensor strip can comprise three layers; a bottom plastic plate 1509 (e.g., about 150 μm thick) for depositing the electrodes; a middle double-sided adhesive layer 1512 (e.g., about 125 μm) for binding to the top and bottom plastics layers and defining the micro-fluidic channel; and a top plastic layer 1515 with two holes or openings for the air in the micro-fluidic channel to escape during the sample fluid flowing into the channel through capillary effect.

As illustrated in FIGS. 4 and 15, the test strip comprises two ends, with sensor electrodes at one end and connector electrodes at the other end. Each set of sensor electrodes (reference and active) can have two exposed contact terminals as sensing areas which can be electrically connected to the connector electrodes at the other end of the strip. To perform the test, the user simply inserts the test strip into the connector port on the handheld device (e.g., similar to a USB plug in) and push a button on the handheld device to start the measurement. An LED light (e.g., red) can indicate that the measurement is in process and another LED light (e.g., green) can indicate that the measurement has been completed, and the test strip can be removed for appropriate disposal. In some embodiments, the test data can be temporarily stored in the handheld device before the data are wirelessly transmitted through, e.g., a Bluetooth® interface to a user device such as, e.g., a smartphone or computer. Once the data is in the smartphone or computer, an application can process the data to inform the user of the test result and give the user an option to upload the data to a secured cloud-based server. In other embodiments, the data can be processed by the user device to provide the test result directly on the LCD display screen as indicated in FIG. 11.

The software application can be developed for user devices such as, e.g., smartphones, tablets, computers or other computing devices. The application can interact with an MCU on the handheld sensor device through a wireless link, e.g., a Bluetooth®, to collect test data, perform simple analysis, and display the test outcomes (positive or negative). The application can also send the detailed test data, with user's consent, to healthcare professionals for further analysis. Wth user's consent, the test data (without user's personal identifiable information) can be collected by a database on, e.g., a secured server. With potentially a large number of users, the large amount of collected data can be valuable for real-time data analytics by researchers to understand the disease epidemiology and help accelerate reopening and economic recovery. The application can be configured so as not to transmit user's personal identifiable information, but only basic demographic and geographic (zip code) information to be associated with the test data.

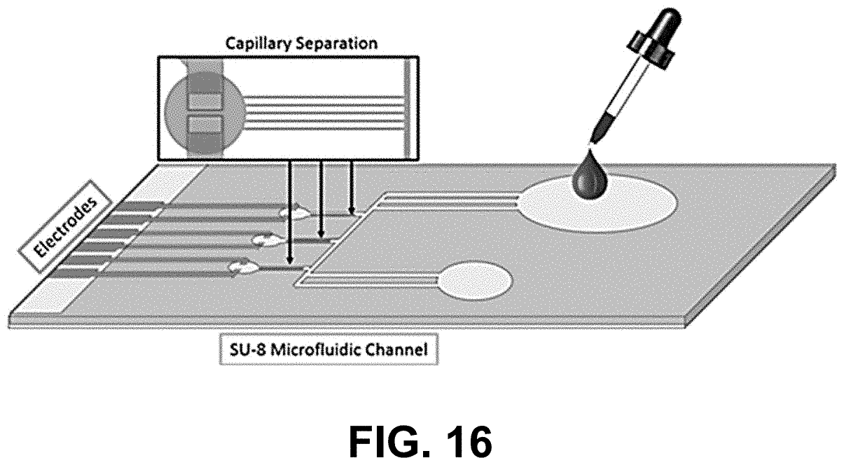

Microfluidics-based component separation can be employed to avoid sample clogging and compromise of separation efficiency, especially for the application of small sample volumes. FIG. 16 is a schematic illustrating an example of a disposable sensor comprising multiple microfluidic channels fabricated on, e.g., a plastic substrate configured with a plurality of sensing electrodes. Test fluids can be loaded in a sample deposit window or opening of the sensor and allowed to propagate down the microfluidic channels to one or more functionalized sensing areas, where it can be analyzed in the sensor device, and the result shown on a hand-held instrument as previously disclosed. In some implementations, separate windows can be provided for each of the functionalized sensing areas.

### Testing Results

Saliva has been shown to harbor SARS-CoV-2 virus at loads comparable to, if not greater than nasopharyngeal secretions, offering a clinical matrix with convenient access amenable to self-collection. To address the current testing shortfall, a novel field effect transistor (FET)-based platform was developed with the ability to detect SARS-CoV-2 spike protein in saliva with exquisite sensitivity, in less than 30 seconds. As disclosed, the handheld system employs disposable sample test strips, offering true point-of-care, non-laboratory sensor technology.

Human saliva has been shown to harbor SARS-CoV-2 throughout the course of infection in levels proportional to systemic viral load and offers painless, convenient access from the oral cavity. The convenience of saliva has been exploited to document disease progression among individuals infected with Zika virus and Ebola during their respective outbreaks. In this regard, SARS-CoV-2 content in saliva can be a more reliable index of viral load among individuals than secretions from nasopharyngeal swabs. The use of saliva as a biological test medium has distinct technical advantages: (1) collection is simple, fast, painless, and non-invasive. Nasopharyngeal swab, in contrast, requires insertion of a long cotton swab deep within the nasal passages, which is manually rotated to collect secretions from the sensitive mucosa. The procedure is not only unpleasant for the test subject but must be administered by trained technicians. (2) Moreover, unlike the nasopharyngeal swab test, the collection of saliva does not induce a sneeze or cough response and thus poses a reduced risk for health care workers, test administers or assistants.

Viral load in saliva has been found to be highest 1-3 days prior to symptom onset and gradually decline thereafter, possibly explaining rapid spread of the virus. Serum antibody against the SARS-CoV-2 spike protein has been detected earlier and more frequently than antibodies against the nucleocapsid. The presence of the virus was examined in 25 patients while correlating the results with comorbidities and levels of lactase dehydrogenase (LDH) and ultrasensitive reactive C protein (usRCP). The drooling technique was utilized to collect saliva, thereby isolating the salivary fluid from sputum or bronchial fluids. It was found that the virus was present in saliva in 100% of the Coronavirus Infectious Disease 2019 (COVID-19) patients confirmed by positive nasopharyngeal RT-qPCR (reverse transcription quantitative polymerase chain reaction).

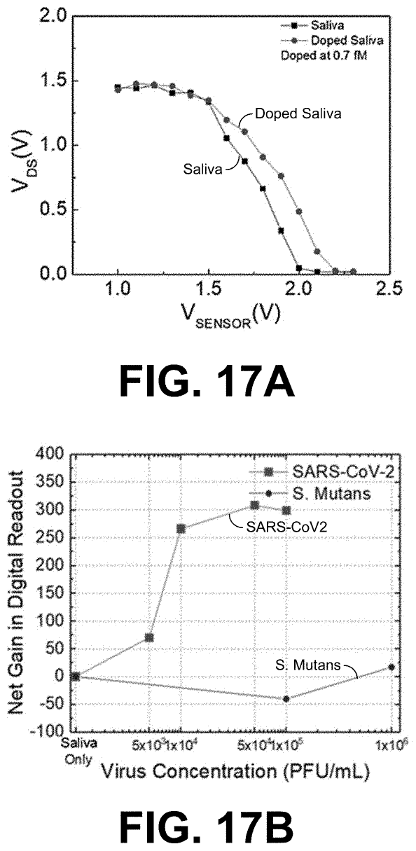

Recombinant peptide corresponding to amino acid sequences found near the n-terminal region of the S2 subunit of the SARS-CoV-2 spike protein, was purchased from ProSci and suspended in PBS at 200 μg/mL. The peptide solution was then serially diluted in artificial saliva (Pickering Laboratories) to obtain the desired concentrations (down to 1 fg/mL). Testing of the sensor platform provided repeatable detection down to the order of 0.7 fM, 1 fg/mL. FIG. 17A shows analog output signals from the testing strip with reference saliva alone and the reference saliva doped with S2 peptide. In order to study the optimal bias conditions for sensitivity, the gate voltage (V_{SENSOR}) was varied from 0.9 to 2.3 V. Relative to the blank reference saliva, a voltage Vₒᵤₜ greater than 0.5 V was achieved in the samples containing the S2 peptide. This graph indicates that the sensor is capable of detecting a signal for the spike protein in very small concentrations that is remarkably different from the signal from the reference saliva.

It was investigated if the strip functionalized with Anti-SARS-CoV-2 antibody could detect SARS-CoV-2 spike antigen when presented on the surface of the virus in the context of human saliva. Heat-inactivated SARS-CoV-2 was employed and diluted in human saliva pooled from multiple donors. FIG. 17B shows an example of the net gain in the digital readout relative to reference saliva, with respect to virus concentration. As shown in FIG. 17B, upon application of saliva spiked with heat-inactivated SARS-CoV-2, the sensor responded to concentrations as low as 5×10³ pfu/mL (or 5 pfu/μL). To investigate the specificity of the detection system, saliva samples doped with increasing concentrations (1×10⁵ and 1×10⁶ colony forming units [CFU]/ml) of Streptococcus mutans (S. Mutants) were exposed to the sensor strip. S. mutans are gram-positive facultative anaerobic bacteria that belongs to a group of mutans streptococci consisting of S. sobrinus and several other species commonly found in the human oral cavity. As illustrated in FIG. 17B, no response was observed to either test sample. These results suggest that the strip sensor has the potential to be used for COVID-19 diagnosis or screening for SARS-CoV-2 infections.

### Characterization of the Readout Integrated Circuit (IC)

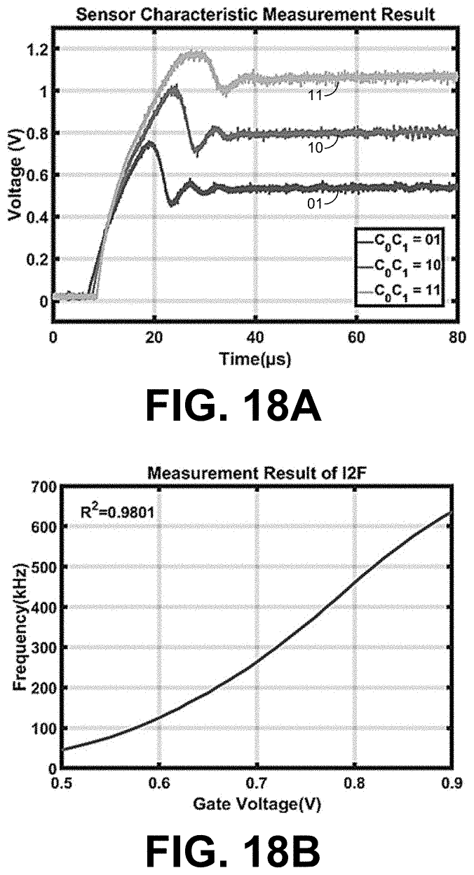

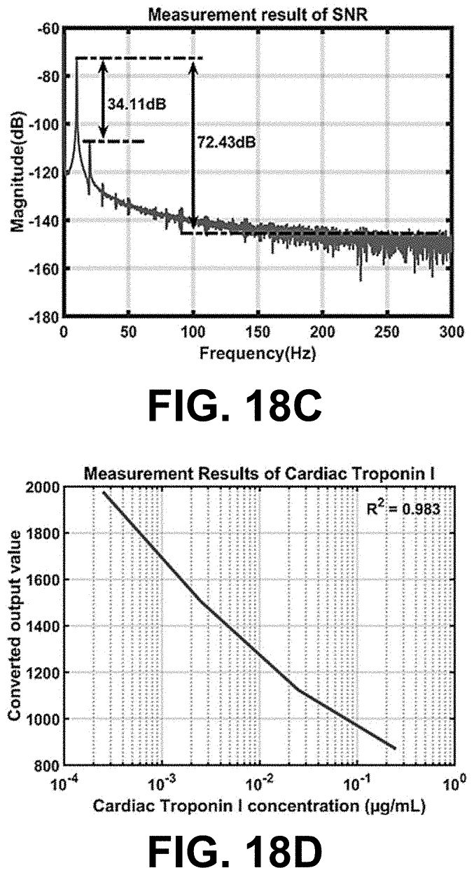

The sensor functionalized for detection of troponin I was tested with the devised readout IC. FIG. 18A shows the sensor output with different pulse voltages (adjusted by C₀ and C₁) in a buffer solution. The settling response can be observed in this measurement. It takes about 10-20 μs for each measurement to stabilize. FIG. 18B shows the measurement results of the I-to-F outputs. As the gate voltage of the MOSFET changed from 0.5 to 0.9 V, and V_{REF} was set to 0.6 V, the output frequency changed from 45.2 kHz to 636.2 kHz. The resulting I-to-F sensitivity was 157.68 Hz/nA, and the measured R² linearity was 0.98. FIG. 18C shows the measured signal to noise ratio (SNR) spectrum of the whole readout circuitry at a 10 Hz sinusoidal input. The spectrum shows the FFT results that were reassembled from the TDC output digital codes by Matlab. The second-harmonics (HD2) was 34.11 dB, and the cross dynamic range was 72.43 dB.

FIG. 18D shows the measurement results of cardiac troponin I while the readout chip was connected with the electrode. Between each measurement of different concentrations of the analyte, the sensor was rinsed with de-ionized water and dried with pure nitrogen for 10 seconds. Then, the troponin I analyte was added to the sensor using a mechanically controlled syringe and the converted output value of the stable state in each measurement was recorded by a programmed data acquisition unit. FIG. 18D shows the converted values at 160 μs after the pulse was triggered in each measurement. The measured R² linearity was 0.983 in the troponin I concentration range of 250 pg to 250 ng with a power consumption of 124.8 μW at 1.2 V. The resulting sensitivity of the proposed system was 1.77 Hz/pg-mL. Generally, in cases of acute coronary syndrome, the peak concentration of circulating cardiac troponin I can reach 0.3-0.5 ng/mL, which is 10-17 times higher than the 99^{th} percentile of the upper limit of the normal reference population. This design makes it possible to detect troponin I quickly and conveniently with minimal power consumption.

By taking ISFET as the reference, a sensor readout circuit with a double-pulse mechanism was developed that is suitable for a variety of sensors. Furthermore, a readout method was implemented using digital circuits, which not only increases the noise tolerance but also prevents analyses from being affected by non-ideal effects on the amplifier. The sensing time for the disclosed sensing system is significantly lower than other cardiac troponin I measurement methods, and there is a reduction in the charge accumulation effects. Wth the high SNR, the detection limit can be as low as 59.76 pg/mL.

This disclosure presents a double-pulsed readout circuit for monitoring SARS-CoV-2, cardiac troponin I, and other biosensing applications. The double-pulse method can reduce the charge accumulation on the channel of the sensing transistor. The signals can be generated from a reconfigurable pattern generator. With the timing interval between two pulses, the sensing path can be reset, reducing charge accumulation on the drain current path and the sensor. When the pulse is applied to the electrode, different concentrations of the analyte induce changes in the gate voltage and the drain current of the transistor. The current information can then be converted by, e.g., an I-to-F converter and a delay-line-based TDC. The measurements show an R² linearity of 0.98 and a sensitivity of 1.77 Hz/pg-mL with 72.43 dB SNR while only consuming 124 μW at a 1.2-V supply. The readout circuitry provides a reconfigurable pulse width, amplitude, and recording window length for a wide range of biosensing applications. The design improves the measurement linearity, sensitivity, and SNR using the time-domain readout technique.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1% to about 5%, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include traditional rounding according to significant figures of numerical values. In addition, the phrase “about ‘x’ to ‘y’” includes “about ‘x’ to about ‘y’”.

Therefore, at least the following is claimed:

1. A medical sensing system, comprising:
a single-use disposable test strip comprising a functionalized sensing area disposed between first and second electrodes, the functionalized sensing area configured to detect SARS-CoV-2 antigen; and
a portable sensing and readout device comprising:
pulse generation circuitry configured to generate synchronized gate and drain pulses, the first electrode of the disposable test strip electrically coupled to a gate pulse output of the pulse generation circuitry; and
a transistor having a drain electrically controlled by a drain pulse output of the pulse generation circuitry, and a gate electrically coupled to the second electrode of the disposable test strip.

2. The medical sensing system of claim 1, wherein the functionalized sensing area is functionalized with anti-SARS-CoV-2 antibody.

3. The medical sensing system of claim 2, wherein the anti-SARS-CoV-2 antibody is bound to a gold (Au) sensing area surface.

4. The medical sensing system of claim 3, wherein the Au sensing area surface is treated with thio-glycolic acid (TGA) to bond the anti-SARS-CoV-2 antibody.

5. The medical sensing system of claim 1, wherein the disposable test strip comprises a second sensing area disposed between third and fourth electrodes.

6. The medical sensing system of claim 5, wherein the second sensing area is a reference sensing area.

7. The medical sensing system of claim 6, wherein the reference sensing area is functionalized with a thiol functional group and without —COOH, —OH or —NH₂.

8. The medical sensing system of claim 5, wherein the second sensing area is a second functionalized sensing area.

9. The medical sensing system of claim 1, wherein the transistor is a Si metal oxide semiconductor field effect transistor (MOSFET).

10. The medical sensing system of claim 1, wherein the transistor can be a field-effect transistor (FET), a bipolar junction transistor (BJT), or their variations in different semiconductor technologies such as silicon MOSFET, GaAs MESFET, InP FET, high electron mobility transistor (HEMT), silicon BJT, heterojunction bipolar transistor (HBT) in SiGe, GaAs, or InP.

11. The medical sensing system of claim 1, wherein the first and second electrodes are Au based metal electrodes disposed on a base structure and covered by a protective cover comprising a sample deposit opening.

12. The medical sensing system of claim 11, wherein the base structure comprises a plastic base strip.

13. The medical sensing system of claim 12, wherein the plastic strip is a polyethylene terephthalate (PET) strip.

14. The medical sensing system of claim 11, wherein the disposable test strip comprises one or more microfluid channels extending between the sample deposit opening and the functionalized sensing area.

15. The medical sensing system of claim 1, wherein the portable sensing and readout device is configured to simultaneously or sequentially test a plurality of sensors coupled to the portable sensing and readout device.

16. A method, comprising:
providing a saliva sample to a functionalized sensing area disposed between first and second electrodes of a disposable test strip, the functionalized sensing area configured to detect SARS-CoV-2 antigen;
generating synchronized gate and drain pulses, the gate pulse provided to the first electrode of the test strip; and
sensing an output of a transistor having a gate electrically coupled to the second electrode of the disposable test strip, the drain pulse controlling the drain connection of the transistor, where the output of the transistor is a function of a concentration of SARS-CoV-2 antigen in the sample.

17. The method of claim 16, wherein the sensing area is functionalized with an antibody specific for SARS-CoV-2 antigen.

18. The method of claim 15, wherein the saliva sample is provided to the functionalized sensing area via an opening in the disposable test strip.

19. The method of claim 18, wherein the opening in the disposable test strip is fluidically coupled to the functionalized sensing area and a reference sensing area by microfluid channels.

20. The method of claim 16, wherein the disposable test strip is configured for single-use, and the transistor is electrically coupled to the first and second electrodes through a detachable connection.
